(19) United States
(12) Patent Application Publication
(10) Pub. No.: US 2014/0257560 A1
(43) Pub. Date: Sep. 11, 2014

(54) BIO-NEURO AND ARTIFICAL INTELLIGENCE CONTROLLED ROBOTIC APPARATUS, SYSTEM AND METHOD

(71) Applicant: Steven Kamara, Princeton, NJ (US)

(72) Inventor: Steven Kamara, Princeton, NJ (US)

(21) Appl. No.: 14/199,275

(22) Filed: Mar. 6, 2014

Related U.S. Application Data
(60) Provisional application No. 61/773,644, filed on Mar. 6, 2013.

(51) Int. Cl. A61F 2/68

(52) U.S. Cl. CPC A61F 2/68
USPC 700/250

(57) ABSTRACT

An apparatus, system and method for controlling a robotic limb. The apparatus, system and method may include at least one microprocessor, a computing memory associated with the microprocessor, the computing memory having resident therein computing instructions, and the computing instructions comprising at least weighting code suitable for weighting control of the robotic limb by the microprocessor versus control by a plurality of bio-neuro sensors associated with the robotic limb. The weighed control may be varied over time by the weighting code.

### CROSS-REFERENCE TO RELATED APPLICATIONS

This application claims priority to U.S. Provisional Application No. 61/773,644, filed on Mar. 6, 2013, which is herein incorporated by reference as if set forth in the entirety.

### BACKGROUND

1. Field of the Disclosure

The present invention relates generally to robotics and, more particularly, relates to a bio-neuro and artificial intelligence controlled robotic apparatus, system and method.

2. Description of the Background

There are several key aspects, and numerous present endeavors, in exoskeleton/bionics/orthotics robotics, medical assistance robotics, and neuro-integrated robotics technologies. One such endeavor is the known, typical electromechanical robotic, which combines mechanical and electrical aspects. Further, for example, modular prosthetics, such as prosthetic legs, may be made available that are purely mechanical, or that combine mechanical aspects with electronic aspects. Such modular electromechanical prosthetics may accordingly allow for robotic actuation of a lost limb, for example. However, even robotic ones of such modular prosthetics typically cannot provide refined control such as that which would be provided by an actual human limb.

One type of the aforementioned technologies is an exoskeleton. An exoskeleton is similar to the modular robotic prosthetic, but is typically placed external to an existing whole or partial limb (rather than being used as a replacement for a lost limb, as would a modular prosthetic) that has become nonfunctional. An exoskeleton, too, comprises mechanical and electrical aspects.

An exoskeleton may serve as a neural prosthesis, i.e., may provide movement in the case of neural damage, to a limb or body part that is still present. Similarly, bionics may serve as neuroprostheses when the limb/part is missing, and may provide movement. These categories of devices may be collectively referred to herein as either bionics or exoskeletons. Further, these devices may enable motor activity for a paralyzed, paretic, or otherwise dysfunctional (i.e., tremors) limb. It should be noted that the terms bionics, exoskeleton, orthotics, and other like terms may be used interchangeably hereinthroughout.

Neororobotic technology, as that phrase is used herein, is thus a robotic element that may include a bio-neuro interface for the robotic element. A bio neurointerface may thus provide sensors that gain insight into brain activity, or insight into nerve activity, with the goal being to replace or enhance typical nervous-system based actuation of a non-functional or lost or partially lost limb. As such, a bio-neuro interface may be employed with an exoskeleton/bionic or a modular prosthetic. Bio-neuro sensors endeavor to allow for a reading of a user's intent to actuate the limb or body aspect that may, at the time of attempted actuation, not be available for full use. By sensing the intent of the user's body, it is hoped that the prosthesis or exoskeleton aspect may be controlled in the same way as would be an actual limb—that is, control of the actuatable robotic occurs by the user's brain attempting to send signals through the nervous system to the nerves of the limb in order that the limb may be actuated. As such, bio-neuro interfaces are intended to provide more refined, human-like motor control to prosthetic limbs and/or exoskeletons.

More specifically, neurorobotic technology may serve to assist patients experiencing a loss or diminution in the body's neuro-connectivity. As such, nerves may be treated, at a base functional level, as electrical connections carrying either output (i.e., motor function) signals or input (i.e., sensory) signals. Accordingly, nerve lesions, or partial or complete nerve severing, whether occurring in the brain, spinal cord, or peripheral nerve, may be viewed as an interruption in signal flow through the foregoing electrical connections, which signal interruption may partially or completely prevent communication in one or both (i.e., sensory and motor) directions.

Thus, neuroprosthetics, which may categorically include bionics and exoskeletons, may connect an artificial device to a still-functioning portion of the user's nervous system. In sum, for example, when a lesion occurs in a peripheral nerve, the distal (i.e., the portion toward the tip of an extremity) functionality may partially or completely fail, but the proximal (i.e., closer to the spinal cord) functionality may still carry detailed motor activity commands. However, due to the interrupt in the “electrical connections,” the detailed motor commands may no longer connect to their respective distal muscles. Consequently, motor commands may instead be directed to an attached bionic device (such as in cases of amputation), or may be reestablished through a neural or pseudo-neural connection, or may serve to control an external exoskeleton (such as in the case of a paralyzed limb or a weakened limb in need of augmentation).

In the particular case of a neural or pseudo-neural connection, nerve sensors may also “record” or “read” remaining healthy sensory signals, such as from a peripheral nerve stump (which provides a richer signal) or from the brain (which provides a weaker signal and which necessitates more invasive techniques to obtain the signal). These nerve sensors in the user's nervous system or brain may thus be used to read the healthy remaining neural signals of the user, which contain the signals for controlling the limb/part, and may thereby enable motor activity, such as by the aforementioned bionics or exoskeleton.

In the alternative case, to enable sensory recovery to damaged distal parts, an artificial sensor may be placed at the distal parts of either a bionic device, such as in the case of artificial fingertips, or, such as in the case of a damaged paralyzed limb, of an external exoskeleton. That is, this distal sensor would be placed on the damaged/paralyzed/numb fingertips, and would then transmit, either via wire or wirelessly, to a separate stimulator at the distal stump of the remaining healthy proximal part of the peripheral nerve, or to a stimulator in the brain itself, such as in the case of spinal cord damage (meaning the brain is the principal functioning proximal nerve point), in order to provide sensory input.

As an alternative to bio-neuro interface control, algorithmic control, i.e., artificial intelligence control, of exoskeleton or prosthetic limbs is being pursued. Artificial intelligence is intended to simulate signals from the brain and nervous system, including the capability to “learn,” and accordingly the intent is to provide refined motor control for prosthetic limbs or exoskeletons. The hope is that artificial intelligence may learn to refine actions over time, just as would the user's brain and nervous system, thereby providing further refined control.

Therefore, the needs exists for a robotic limb or other body part replacement or assistance apparatus, system and method, that is suitable to provide body part replacement or assistance having the refined control of the user's nervous system, and in addition the “learning” capabilities of an artificial intelligence system.

### SUMMARY OF THE DISCLOSURE

The present invention is and includes an apparatus, system and method for controlling a robotic limb. The apparatus, system and method may include at least one microprocessor, a computing memory associated with the microprocessor, the computing memory having resident therein computing instructions, and the computing instructions comprising at least weighting code suitable for weighting control of the robotic limb by the operating code for the robotic limb versus control by a plurality of bio-neuro sensors associated with the robotic limb. The weighed control may be varied over time by the weighting code.

Thus, the present invention provides a robotic limb or other body part replacement or assistance apparatus, system and method, that is suitable to provide body part replacement or assistance having the refined control of the user's nervous system, and in addition the “learning” capabilities of an artificial intelligence system.

### BRIEF DESCRIPTION OF THE FIGURES

The disclosure will be described in conjunction with the following figures, in which like numerals represent like elements, and in which:

FIG. 1 is a flow diagram illustrating a weighted control system according to the present disclosure;

FIG. 2 is a graphical illustration of aspects of the present disclosure;

FIG. 3 is a schematic diagram representing aspects of the instant disclosure; and

FIG. 4 is a schematic diagram illustrating aspects of the disclosure.

### DETAILED DESCRIPTION

The figures and descriptions provided herein may be simplified to illustrate aspects of the described embodiments that are relevant for a clear understanding of the herein disclosed processes, machines, manufactures, and/or compositions of matter, while eliminating for the purpose of clarity other aspects that may be found in typical robotic devices, systems, and methods. Those of ordinary skill may recognize that other elements and/or steps may be desirable or necessary to implement the devices, systems, and methods described herein. Because such elements and steps are well known in the art, and because they do not facilitate a better understanding of the disclosed embodiments, a discussion of such elements and steps may not be provided herein. However, the present disclosure is deemed to inherently include all such elements, variations, and modifications to the described aspects that would be known to those of ordinary skill in the pertinent art.

As mentioned above, replacement limbs or limb assistance are typically provided in two types—robotic prosthetic limbs and exoskeleton/bionics limbs. Robotic prosthetics are clearly defined, and include a substitute for a user's limb, which substitute does not, itself, include aspect of the user's nervous system. That is, the user's nervous system may, using the aforementioned aspects, be connected, such as via bio-neuro sensors, to electronic circuitry in the robotic prosthetic.

The second type of limb replacement or assist may include an exoskeleton. An exoskeleton (which may be used herein to include bionics) may be placed external to or partially implanted in a user's body, and is typically employed in conjunction with a full or partial limb remaining on the user's body. As such, exoskeletons may be used to treat patients suffering from paralysis, such as from an accident or stroke, diabetic patients, patients suffering nerve or spinal injuries, patients suffering from benign tremors, Parkinson's disease, or the like, and patients experiencing old age, wherein the patient loses strength and/or dexterity.

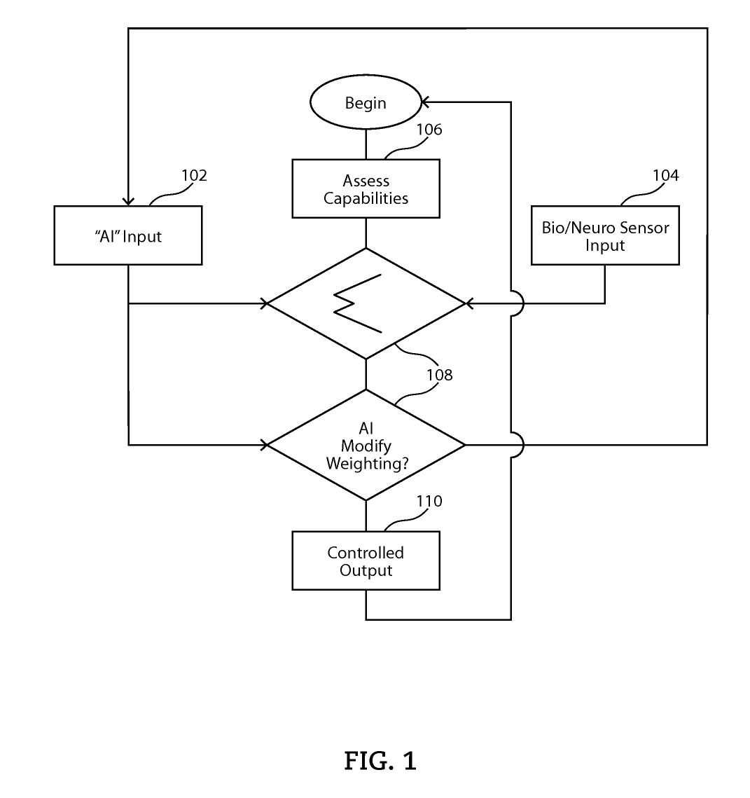

In the case of prosthetics or exoskeletons/bionics (which may be collectively referred to with regard to aspects of the disclosure herein as “exoskeletons”), the typical approach to a control system varies to either developing artificial intelligence, or endeavoring to amplify actual nerve signals based on nerve signal sensing. In contrast, and as illustrated in the flow diagram of FIG. 1, the control system of the present invention incorporates both artificial intelligence at step 102—that is, learned or programmed behavior—and nerve signal sensing at step 104, along with sensor-assessed capabilities and/or environment at step 106, in a weighted manner at step 108 that varies the weight of output control 110 accorded to the artificial intelligence versus the nerve signal sensing based on any number of factors. For example, a user recovering from a spinal injury may experience increased nerve function, and thereby the weighting for an exoskeleton limb assist may increase in favor of nerve signal sensing as time wears on and as the user heals. In such embodiments, an exoskeleton assist may be principally used for a variety of reasons, not the least of which is the noninvasive nature of the use of an exoskeleton.

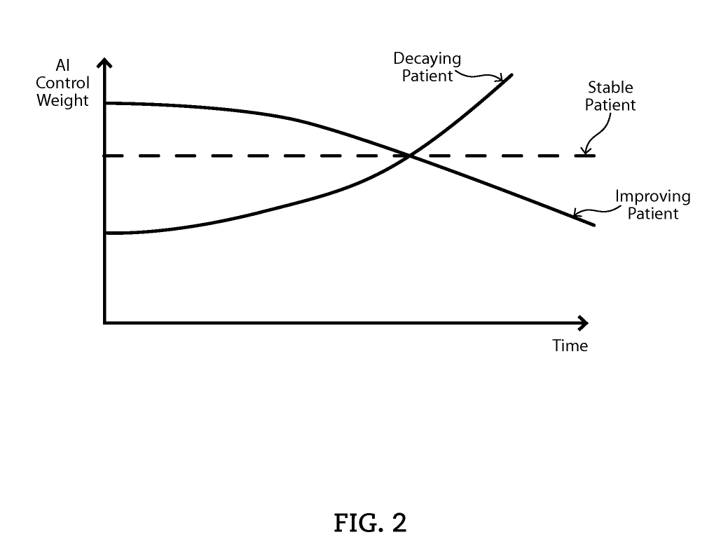

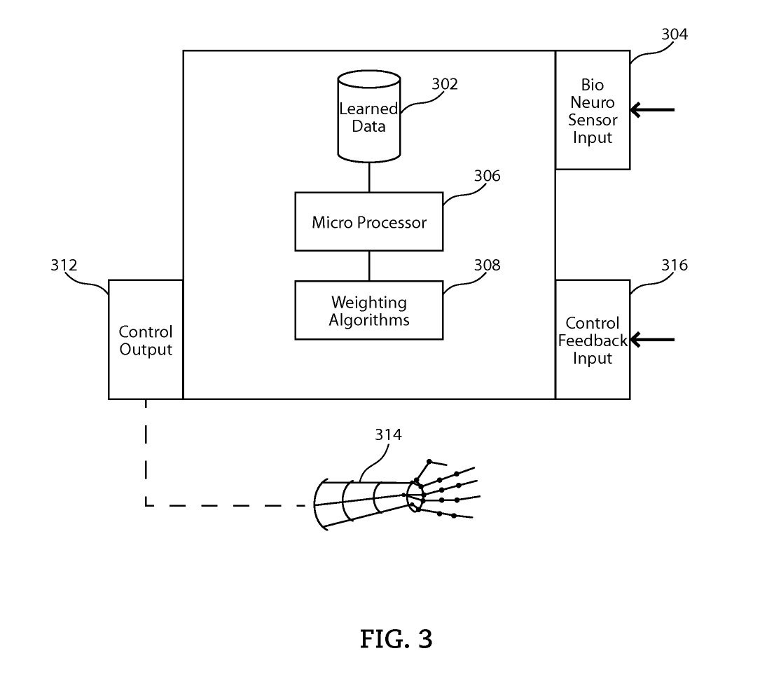

As such, a weighting system in accordance with the present invention may be tailored to a user's level of disability, and may vary over time, as illustrated graphically in FIG. 2. According to this time-based control, one or the other aspect—that is, the artificial intelligence versus the nerve sensing—may be granted control over changes in the weighting process. Most preferably, it is the artificial intelligence, due to its learning capability and its consequent association with a microprocessor 306 and computing memory 302 that are suitable for algorithmic modification, that would control the weighting system 308 as between it and the nerve sensing system. Accordingly, the artificial intelligence may learn and accumulate learned data 302 over time, and thereby allow for refined movement, but may additionally monitor for increased nerve sensing control 304, at which point in time the artificial intelligence system may compare its own suitability for refined motor control to the increasing suitability of the user's bodies own nervous system for fine motor control, and may weight control accordingly. Such a modifiable, microprocessor-driven system is illustrated in the schematic illustration of FIG. 3.

The microprocessor 306 may receive input, i.e., sensory data, from any of a variety of sources. For example, input for weighting control may not only be received from the user's neurodata 304, but may additionally include, for example, surrounding data input within view, such as may be assessed via a mobile device, Google glasses, or the like. Moreover, neuro-sensory data may be produced to enable motor activity from so-called “emotiv-type”, EEG personal external noninvasive brain sensors. Yet further, sensory information may be gathered using a technique called “optogenetics,” which may enhance “mapping” of subspecific sensory and motor data from either the brain or peripheral nerves or senses of patient. Of course, those skilled in the art will appreciate that any type of available sensors may be used, such as flexible metallic, transduction, rubberized, plastic, or like sensors, which may reside within the body, such as brain proximate or peripherally within the nervous system, or without the body.

Further, and with continued reference to FIGS. 1, 2 and 3, control of output 312 provided in the artificial intelligence system for exoskeleton parts 314 may allow for various other atypical controls by the user. For example, the user may employ voice dictation 316 in order to improve action indicated by the artificial intelligence, such as in conjunction with mechanical control data/feedback data 316. For example, a user may indicate to an exoskeleton arm that a cup is to be picked up at three o'clock from the user. The artificial intelligence may be programmed to, or over time may have learned to, employ only that pressure which is necessary in order to pickup the cup at three o'clock. Further, the artificial intelligence may be capable of moving the hand of the user outward until sensors in the exoskeleton, or the user's nervous system (bio-neuro) sensors, reach the sensation of having touched the cup, whereafter the artificial intelligence may instruct that the hand be closed about the cup, either independently or in a weighted conjunction with the referenced nervous system sensors. As such, the weighted system may be employed wherein artificial intelligence and nervous system sensing are simultaneously employed, and additional controls may be granted to the user in a microprocessor-driven artificial intelligence system.

Similarly, in an artificial intelligence controlled system, a user may be provided with a verbal override, whereby a user can stop, start or modify an action based on voice control. For example, a user may be provided with a Bluetooth headpiece, or a Bluetooth earbud, wherein the Bluetooth is linked to an exoskeleton arm assist. Thereby, the user could use voice controls and/or voice commands to control exoskeleton aspects linked to the Bluetooth. Thereby, at least through the use of a safety override, the present invention provides improved safety of use over the available art.

Additionally, and by way of non-limiting example, output functionality may change for a single input method when a different situation demands different output functionality. For instance, in the aforementioned voice-controlled embodiment, a voice activated microphone input system may need to control a user's left bionic upper extremity limb (such as with a set of understood commands like “up, down, slower, stop,” etc.), but the same commands coming from the same voice activated microphone may also need to control the user's right limb in another scenario. Likewise, the signals from a single small motor cortex brain sensor may provide the ability to control two or more peripheral functions (such as by controlling three different parts for a stroke patient, such as by receiving his left leg intentions, his left hand intentions, and his vocal cord intentions). The weighting algorithm discussed throughout may be optimized for use in such scenarios, such as by making the control language input set large enough so that there is no language overlap for different desired simultaneous functions, or by using a word modifier could be used to change the desired output limb that would be activated (“left leg stop”, “right wrist flex slowly”, or “right elbow extend slowly B mode”, whereby “b mode” signifies using a high-strength mode vs a low strength mode for the specific movement), and wherein use of the control language automatically executes not only the instruction, but a switch to proper weighting to carry out the directed function, upon receipt of the voice command.

Additionally, other atypical control methodologies may be provided for use with the weighting system of the present invention, and/or for control over or modification of such weighting. For example, applications, also-referred to as “apps”, for mobile devices may be provided and enabled with control features for the aspects hereof. Such apps may be provided for unique components, global components, or the like, of an exoskeleton system, and may be modifiable by a user or may require an administrator (i.e., medical personnel) level access for modification.

A weighting system, which may or may not include voice control as an input (such as in conjunction with other input, such as neuro-sensors), for example, may further provide weighted control based on use circumstances, such as a need for strength versus a need for dexterity. For example, picking up a china cup would require extreme dexterity and very little strength, but picking up a fireplace log would require significant strength and very little dexterity. Accordingly, the artificial intelligence discussed herein may be aware of the user's nervous system's capability for strength, or dexterity, and the artificial intelligence may provide added weighted control in whichever category the user's nervous system suffers from more. Moreover, such a weighting system, in conjunction with the aforementioned verbal technologies, may allow for a user to verbally tune, such as for dexterity or strength, in any given application, or over time.

Additionally, an exoskeleton or a prosthetic in accordance with the present invention may additionally include optical technologies, such as those referenced above, whereby a robotic limb may recognize shapes, size, or the like, either on-limb or via a mobile device or Google glass connection, and which visual recognition system may additionally be available with the aforementioned voice commands. As such, the artificial intelligence system discussed herein may learn via any of a variety of sensors, and may adapt and re-weight control over time based on the user's capabilities, in a manner akin to a learning “auto correct” for a specific user's frequent typing in a word document program.

Other aspects may be provided in the present invention. For example, a prosthetic or exoskeleton may be provided to a user lacking all or a portion of a limb, wherein such exoskeleton or prosthetic may be programmable via the aforementioned Bluetooth, or via an RF interface, or via a NFC interface, or via other known methodologies, within the prosthetic or exoskeleton. Further, in such embodiments, a realistic looking “skin” that is electrically conductive may also be provided in conjunction with such a prosthetic limb or exoskeleton. Such a skin may act in a manner akin to real skin, and as such may have the proper capacitance characteristics to enable an interface with, for example, a smart phone screen.

More particularly, a subject with partial movement may have her movement level assessed. A noninvasive exoskeleton technology in accordance with the present invention may, based on this assessed level, provide the artificial intelligence information to increase an amplitude of nervous system sensing already present in the user, and may receive information that the artificial intelligence is to have “master weighting,” such as 80% weighting, to instruct as to direction of movement of the limb. As such, the weighting discussed herein may take an inertia or a minor indication indicated by the nervous system of the user, and may augment strength, direction, stability, or the like.

Further, it should be noted that weighting algorithms employed may vary in application to the present invention, as will be evident to those skilled in the art in light of the discussion herein. For example, an artificial intelligence system may begin, for a patient known to have limited, but some, nervous system indication to a limb, at 90% artificial intelligence control and 10% neuro-bio sensor control. However, when bio-neuro indications are that availability natural neural signaling reaches a level 2 (such as on a 1 to 10 scale), bio-neuro signaling may be amplified, and the amplified signal accorded a weight of 25% control—thus meaning that the artificial intelligence control would drop to 75%.

Such “artificial intelligence” learning systems are applicable to all aspects of the instant disclosure. The use of such artificial intelligence may be enhanced not only through “learning” the user's needs and desires, but additionally though use in conjunction with a “rewards” system. That is, a user may receive an inadequate weighting for the help actually need to, for example, move her exo-arm, in part to drive the user to work harder to use the subject exo-arm. Accordingly, as the user practices and better learns to use her exo-arm, the artificial intelligence will “see” the user's enhanced efforts, and will increase the weighting algorithm to provide adequate weighting for the robotic aspects of the arm so as to provide to the user full use of the exo-arm.

As referenced throughout, sensory inputs, such as from more than one source, may be weighted, balanced, or otherwise independently contributed to a decision, by the one or more microprocessors, to produce a movement, i.e., a motor output. Exemplary inputs, in addition to those specifically set forth and discussed herein, may include positional sensors, velocity sensors, accelerometers, ultrasound, gyroscopic sensors, optical sensors, “computer vision”, infrared, tactile sensors, audio microphones, eye positional sensors, tongue positional sensors, and any other inputs known to those skilled in the pertinent arts.

The signal output in accordance with the computations of the microprocessor(s), which may be referred to throughout as a motor output, may comprise a direct-drive output for an exoskeleton, for example. Further, for example, synthetic muscles may be driven by the output signal(s) in conjunction with the more typical electrically-powered motored movements, such as to optimize an outcome for situational and/or desired motor goals (for example, more endurance may be needed in certain situations, while more speed may be required in others; more dexterity may be needed at times, while speed is needed at other times; more quiet operation may be needed at times, while more powerful operation may be needed at other times; and the foregoing alternatives may have inverse relationships, such as may be mathematically computed by the processor according to the afore-discussed weighting).

Further, output signals may comprise multiple outputs, such as to various systems or to various components of a system. Such multi-outputs may be provided wired or wirelessly, or via combinations thereof. For example, wireless data communication may be provided between various components of an exoskeleton embodiment based on specific patient needs. More particularly, for example, wireless communication may enhance neural feedback systems among all or some input sensors and/or output methods, including combined subvariations. For instance, communication between brain sensors, brain stimulators, peripheral nerve sensors, and peripheral nerve stimulators for a patient whose bionic system makes use of all four elements may be corresponded wirelessly between sensors, and with a microprocessor executing the weighting for output discussed throughout. By way of non-limiting example, the interface of a peripheral nerve stimulator to a peripheral neural interface (such as a peripheral nerve cuff or piercing intrafascicular electrode) may occur wirelessly. Also, a wireless connection may facilitate communication between peripheral nerve sensors and a traditional spinal cord stimulator, such as to enhance sensory function (whether touch or proprioception functionality), or to enhance pain reduction, and/or to increase blood circulation to the areas distal to nerve lesions.

The present invention may additionally provide enhanced output control of complex multi-limb exoskeleton systems. The more separate deficits the user has, that is, the more limbs or parts that require exoskeleton assistance or replacement for function, the more complex the communication, and hence the weighting and balancing, as between and among the inputs and outputs. As discussed herein, the use of wireless communications for at least some aspects of this communication may simplify otherwise complex intra- (and inter-) system communications. Further, specific control strategies/algorithms are required, especially for such more complex bionic/exoskeleton systems, and/or as additionally deficiencies and/or peripheral nerve deficits arise in the user, and the presently disclosed weighting and balancing system allows for such specific control algorithms, and for the targeting of such specific algorithms to individual components in a manner tailored to the user's needs with respect to that part.

In the manner discussed throughout, the weighting system may provide output to control, for example, the “angle” of a prosthetic joint (such as in case of bionic limb), the “range” of the actual limb (such as in the case of an exoskeleton which moves the limb, or assists in the movement of the limb), or like limitations stemming from the output of the weighting system, or from an operating mode into which the weighting system is placed. Changing or limiting the range of a joint, may, for example, provide a safety or optimization feature for a better experience based on the situational activity of the user (for instance, in a “heavy duty lifting” mode, the range of a joint may be restricted, so as to prevent an injury of a prosthetic or real joint). Similarly, in a “dexterous activity” mode which may be selected for activities like preparing food or playing an instrument, the joints should have maximal range, since such a mode might include a reduction of operating forces.

In short, the weighting system disclosed herein may provide a plurality of operating “modes” that may be selected based on input sensing by the CPU and/or the weighting system, the type of activity the user is engaged in, or by the user or an administrator, and that may affect the type and manner of the output to the exo-part. Of course, in certain cases, a user may wish to select a particular mode that is not “typical” for his current activity, but that may provide a certain desired effect, and such an atypical mode may have a unique risk/reward profile. Accordingly, the weighting system may “ease” the user through the new mode, may alert the user to the atypical mode choice, or the like. Such a mode-based output system may be similar to the manner in which a digital camera has an “auto” mode that automatically selects a mode based on the camera's sensor inputs, and a manual mode in which a user can chose “landscape”, “portrait”, or like modes, based on his objectives, whether or not those objectives are atypical.

Such a mode-based output system may comprise controls for speed of limb/limb parts, acceleration of the same, absolute positioning of the same (for instance, in a eye-following mode for a hand, sensing of the absolute position of the eye and a direct translation to limb-movement limits), user alerts, safety features, etc. Also, mode selection may be weighted by the weighting system discussed herein, wherein a mode or modes may be tailored to provide more suitable automatic mode(s) based on the calculations and learning provided by the CPU and weighting system hereinthroughout. Needless to say, manual weighting and mode operation are viable in all cases, but it should be noted that such manual operations may be more cumbersome for real-time changes to operation.

The intuitive weighting and artificial intelligence system, and its modes, as disclosed herein may operate in a manner similar to how able-bodied persons change their respective “modes” based on certain factors (e.g. if a lot of a person's body is engaged in strong forceful motions, such as exercise, it is in “high force” mode and as such may make other joints comply with this “mode”). Of course, sometimes an automatic pre-set predictive mode system won't operate according to the entire set of user's desires (such as wherein a user wants to exercise her exo-legs, but use dexterous movements in the bionic/exoskeleton hands to control a mobile phone), and in such cases the artificial intelligence and weighting may allow the user to actively train the system to act differently next time those simultaneous circumstances arise, or the CPU and weighting algorithm may recognize that the effect was bad through the input sensors, and as such may modify the approach the next time. Of course, like combinations may arise within various sub sets of one or more particular body parts, such as between the 3 phalanges of the finger or the five fingers of the hand, or the hand, wrist, elbow of the entire arm, by way of non-limiting example.

For example, an output mode modification, based on an input modification, in the present invention may include a haptic sensory transfer system (which may feel vibrations, pressure, or the like). For example, if artificial finger tips get stimulated neutrally, if the user's sensory input system fails and cannot be artificially restored (since, for instance, the sensory stimulator cuff, or any other neural interface, became electrically disconnected from the peripheral nerve, or from the brain, from which it is to take direction), but the motorized parts of the bionic/exoskeleton system remain intact, alternative input, such as visual or voice input, may be switched to in order to restore control and movement of robotic parts.

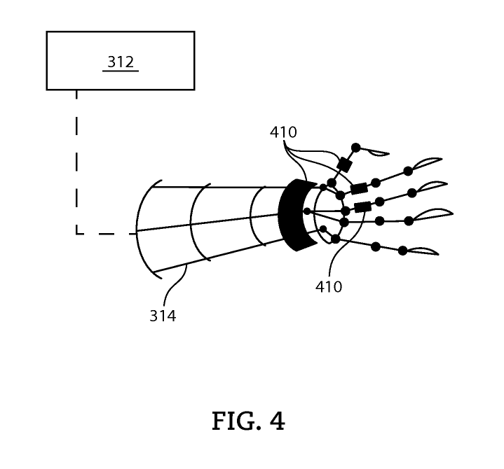

Yet further, dynamic/flexible orthoses, or braces, may be provided as “extensions” 410, such as is illustrated with respect to FIG. 4, and may receive output and control 312 to enhance bionic/exoskeleton part 314 functionality. Such dynamic braces may provide a variety of enhancements to output functionality as needed, such as to enhance functional power, endurance, dexterity, or safety, among other possibilities, such as by blocking or tapering unwanted specific motions or power or speed of motions, by providing enhanced stability, and/or by strengthening weaker desired movements (i.e., complete or partial deficits) through energy transfer. Such dynamic “splints” include, among others and by way of non-limiting example, ankle foot orthoses, which may be used for “foot drop” from central or peripheral origins.

Thereby, the present exemplary embodiments may provide not only a combination of non-powered dynamic splints with powered bionics, but also may provide a new type of dynamic splint. For example, a dynamic finger splint may create desired enhancements of the specific type of intrinsic hand muscle motor function that is most in deficit (individual finger flexion movements, finger extensor movements, or finger abduction and finger adduction movements, by way of non-limiting example).

In yet additional exemplary embodiments, surgical enhancements may be provided to allow for optimized motor output in the disclosed systems and methods. For example, in cases of amputations and peripheral nerve damage, surgical peripheral “nerve transfers” may be combined with enhancements of a provided bionic to optimize results. Peripheral “nerve transfer” is generally accepted to sacrifice a less important or useful healthy donor peripheral nerve or nerve fascicle in order to enhance a more important, but damaged/non- or limited functional, nerve or nerve fascicle, to thereby enhance motor output functionality.

Yet further, the instant invention may include the ability to non-surgically upgrade the aspects discussed herein. For example, all external or embedded controllers may be enabled for wireless upgrade and wireless upload.

The output from the system herein may also serve to “plug and play” with additional devices that may serve as alternative “extensions” of the exoskeleton systems disclosed herein, such as those referenced above with respect to FIG. 4. For example, special mouse and keyboard devices made specifically for specify bionics/exoskeleton. Similarly, the weighting herein may be varied by extension and setting. For example, a car may be exo-equipped, and the user's settings may be unique to his car. The weighting settings may thus download, such as from the cloud, the exoskeleton itself, or another local device, either upon each use or upon first use by the exoskeleton user. Accordingly, the weighting herein may be local or cloud-based, and may be stored locally, in the cloud, or at a discrete extension, i.e., on-board the car in the above example. Thereby, weighting may be varied by the setting—for example, the exoskeleton (or a corresponded device, such as a mobile device), may sense the setting (such as using G.P.S., triangulation, mapping apps or programs, frequent destination, wireless network identification, or the like), and may download/upload the indicated parameters in view of the weighting data/algorithm assigned for that setting and/or for that extension.

Needless to say, the instant invention may additionally include a plurality of safety features to enhance the operability of the invention. Such safety features may be treated by the code as extensions, as discussed elsewhere herein. For example, the invention may comprise hard mechanical safety features, such as a magnetic system for modular prosthetic bionic limbs or exoskeletons. In such a system, if too much force is applied, the magnetic safety feature disengages the robotic element, in a manner similar to that in which a magnetic power cord may “snap” out of a laptop socket if too much force is applied to the pulling of the cord.

Likewise, the instant invention may include one or more non-mechanical safety features. By way of non-limiting example, the weighting methodology described herein may assess that the bionic/exoskeleton limb senses is in danger (i.e., is suffering from possible self-destructive or externally-destructive mechanical forces), the weighting algorithm may automatically engage a reversal/retraction movement, or a slowing of pace, or a complete halting of action, etc.

By way of additional non-limiting example of safety features, as set forth herein voice may control some aspects of the actions of the CPU of the bionic/exoskeleton, and in such embodiments the voice control may accept and respond only to the user's singular voice properties (such as may be assessed through the wireless microphone), and/or to an administrative voice (such as a doctor). Similarly, a highly sensitive microphone may accept only voices coming from within the field of the microphone (in a manner similar to some cell phones). Such mechanisms may, of course, be performed as part of the weighting code, and as such the system disclosed herein may select whichever voice control method proves safest, is most often correct in its perception, and/or is more resilient.

Although the invention has been described and illustrated in exemplary forms with a certain degree of particularity, it is noted that the description and illustrations have been made by way of example only. Numerous changes in the details of construction, combination, and arrangement of parts and steps may be made. Accordingly, such changes are intended to be included within the scope of the disclosure, the protected scope of which is defined by the claims.

What is claimed is:

1. A system for controlling a robotic limb, comprising:
at least one microprocessor;
a computing memory associated with the microprocessor, the computing memory having resident therein computing instructions comprising operation instructions; and
the computing instructions comprising at least weighting code suitable for weighting control of the robotic based on the operation instructions versus control by a plurality of bio-neuro sensor data perceived by a plurality of sensors associated with the robotic limb;
wherein the weighed control is varied over time by the weighting code.

2. A system for controlling a robotic limb, comprising:
a robotic limb comprising at least one microprocessor for controlling a first wireless connection;
a computing memory in communication with the microprocessor, the computing memory having resident therein computing instructions; and
the computing instructions comprising a received at least weighting code suitable for weighting control of the robotic limb based on corporal functionality versus coded operating instructions for the robotic limb;
wherein the operating instructions are received via the first wireless connection.

3. The system of claim 2, wherein the corporal functionality is assessed by a plurality of sensors associated with the robotic limb.

4. The system of claim 2, wherein the computing instructions are received by the first wireless connection.

5. The system of claim 4, wherein the computing instructions are resident in association with a second microprocessor remote from the first microprocessor.

6. The system of claim 2, further comprising a second robotic limb, wherein the second robotic limb receives second computing instructions, and wherein the second robotic limb communicates with the robotic limb via the first wireless connection.

7. The system of claim 2, wherein the computing instructions further comprise a learning module suitable for modifying the weighting code over time.

8. The system of claim 7, wherein the learning module modifies the weighting code according to the corporal functionality.

9. The system of claim 2, wherein the robotic limb further comprises an extension.

10. The system of claim 9, wherein the extension comprises a dynamic splint.

11. The system of claim 9, wherein the extension comprises a computing input.

12. The system of claim 2, further comprising a plurality of sensors that provide information regarding the corporal functionality.

13. The system of claim 12, wherein at least ones of the plurality of sensors are implanted.

14. The system of claim 2, further comprising a plurality of sensors for sensing an environment in which the robotic limb is present.

15. The system of claim 14, wherein ones of the plurality of sensors comprise optical sensors.

16. The system of claim 14, wherein ones of the plurality of sensors comprise geographic location sensors.

17. The system of claim 2, wherein the coded operating instructions are uploaded via the first wireless connection.

18. The system of claim 2, wherein the computing instructions further comprise control instructions suitable for acting on the coded operating instructions.

19. The system of claim 18, wherein the control instructions are indicated by voice control communicatively associated with the robotic limb.

20. The system of claim 18, wherein the control instructions are indicated to a mobile device.
